(19) United States
(12) Patent Application Publication
(10) Pub. No.: US 2015/0068270 A1
(43) Pub. Date: Mar. 12, 2015

(54) Background Cancellation with Electronic Noses

(71) Applicants: Royce W. Johnson, Universal City, TX (US); Alexei Tikhonski, Cedar Park, TX (US)

(72) Inventors: Royce W. Johnson, Universal City, TX (US); Alexei Tikhonski, Cedar Park, TX (US)

(73) Assignee: Applied Nanotech Holdings, Inc., Austin, TX (US)

(21) Appl. No.: 14/395,412

(22) PCT Filed: Apr. 24, 2013
(86) PCT No.: PCT/US2013/037944

§ 371 Date: Oct. 17, 2014

Related U.S. Application Data
(60) Provisional application No. 61/638,100, filed on Apr. 25, 2012.

(51) Int. Cl. G01N 33/00; G01N 35/00

(52) U.S. Cl. CPC G01N 33/0008; G01N 35/00594
USPC 73/1.06

(57) ABSTRACT

A method and apparatus for background cancellation for electronic noses to make automated aroma analysis practical in complex field environments. The system and methods compensate for background contaminants while automatically emphasizing all constituents, be they chemically identified or not, which represent information content in the sample being tested.

This application claims priority to U.S. Provisional Application Ser. No. 61/638,100, which is hereby incorporated by reference herein. This application is related to U.S. Provisional Patent Application Ser. No. 61/583,288 and U.S. Published Application No. 2013/0066349, which are hereby incorporated by reference herein.

### BACKGROUND INFORMATION

Automated detection of aromas has been achieved with limited success using a class of technology known loosely as “e-nose” instruments. These instruments (e.g., the Cyranose commercially available from Cyrano Sciences) employ some form of a sensor array to measure the presence of volatile organic compounds in a gaseous sample. To apply such analyzers to detect the presence of some targeted condition (e.g., an infection in a wound or contamination in a food stock) requires that the components of the condition's aroma signature be known and then gas sample analysis(es) performed to compare the sample's signatures to the known signature.

The technology has only been successful in controlled laboratory environments at least for two reasons. The first reason is that the devices generally operate with a limited, and fixed, number of chemical detectors, each of which must be preselected by fore-knowledge of the chemical composition of the anticipated aromas. This limits the technologies to aromas that are either simple in composition or stable over time. The second reason is that laboratory conditions allow for excluding any confounding background odors from the analysis environment simply by limiting the presence of odor-producing materials. This is clearly not the case for field conditions where odor-producing materials are ubiquitous. The two issues are exacerbated by the use of highly sensitive chemical detectors capable of measuring very small amounts of volatiles in the sample, leading to over emphasis of extraneous compounds in the response or saturation of the detectors when large quantities of their analytes are present.

The consequence of these issues is that c-nose technologies have not been successfully applied to a full range of applications that may be amenable to detection by automated aroma detection. Of particular interest to society are healthcare applications, but these are also the most challenging fur at least the two reasons previously set forth. The multiplicity and time-varying nature of pathophysiologic states, patient co-morbidities, and pharmacologic interventions, which are present in all seriously ill patients, make the targeted aroma signatures very difficult to predetermine. Similarly, the complexity and inter-site variations in environmental aromas make the aromatic signal-to-noise ratio especially challenging.

A publication by Jane Hill and colleagues (see, Jiangjiang Zhu, Heather D. Bean, Yin-Ming Kuo, and Jane E. Hill, J. Clin. Microbiol., 48 (12): 4426-4431, 2010) illustrates the problem when their supplemental material is ethically examined. FIG. 1A shows a reproduction of their original data in the supplemental file. It is clear from this table that the large majority of compounds present in a typical headspace sample are not easily identifiable. FIG. 1B shows their exemplar SESI-MS plots in full detail and curve-averaged over nine gas samples. The fact that many individual scans were required to produce these complex plots indicates the underlying complexity and inter-sample variability typical for biomedical applications. And note as well that these plots were obtained under laboratory conditions and presented after the background spectrum of the growth medium had been subtracted. This represents a nearly ideal case where a known, stable background aroma was present.

### SUMMARY

Embodiments of the present invention provide conjoint improvements to make automated aroma analysis practical in complex field environments. The promise of automated aroma analysis has never been fully achieved because of the issues of background constituents confounding the limited analytical ranges of fixed-sensor electronic nose technologies. Recognizing that conventional electronic nose technologies utilizing arrays of single-compound sensors are both sensitive to background contaminants and miss as tremendous number of unidentified but potentially didactic constituent compounds in the complex aromas of field samples, described is a novel system of apparatus and methods that compensate for background contaminants while automatically emphasizing all constituents, be they chemically identified or not, which represent information content in the sample under test.

### BRIEF DESCRIPTION OF THE DRAWINGS

FIG. 1A shows a table of SESI-MS positive ion-mode peaks listing for four species of clinically significant bacterial pathogens (aeruginosa (P.a.), S. aureus (S.a), E. coli (E.c.), and S. typhimurium (S.t.) in vitro culture after 24 hours growth in TSB at 37° C.

FIG. 1B shows a positive ion-mode SESI-MS spectra of bacterial culture headspace for S. pullorum and S. typhimurium grown aerobically in TSB at 37° C. for 24 hours.

FIG. 2 schematically illustrates a GC-DMS analyzer.

FIG. 3 shows an example of an odorgram.

FIG. 4 schematically illustrates adaptive removal of odor noise from an analysis result of a combined gas sample.

FIG. 5 schematically illustrates sampling of a solution with multiplexing through a single analyzer.

FIG. 6 schematically illustrates use of SPME fiber to sample historical exposure to en environmental chemical constituents.

### DETAILED DESCRIPTION

A solution to the foregoing involves three conjoint improvements to the practice of the current art in e-nose methods. A first improvement is to spread the chemical signature analysis into at least one additional dimension to create a two-dimensional (“2D”) odorgram. This confers a benefit of a much more sensitive and specific data set to operate upon. A second improvement is to recognize that the data set is generally an unknown mixture of signal and noise that must be separated by using a noise reference: this may be accomplished here with adaptive noise cancellation algorithms. The science of odor analysis has been so focused on identifying individual chemical analytes in the odor profiles that the question of whether the intervening peaks in a spectrum represent signal or noise has never been effectively investigated. Kwak and Preti (see, Jae Kwak and George Preti, Current Pharmaceutical Biotechnology, 12:1067-1074, 2011) raised the specter of an irreconcilable admixture of signal and noise constituents in odor signatures and implied that it was an intractable problem. It is not. A third improvement is a means to obtain a reference source of merely the contaminating odors, which need be only similar, not identical, to those contaminating the sample itself.

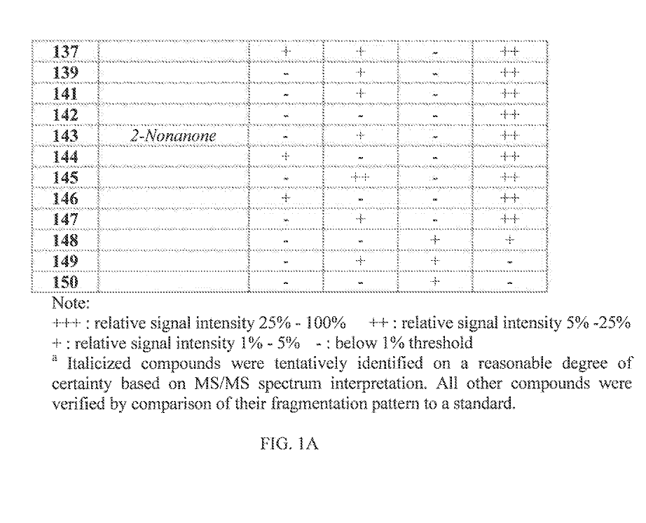

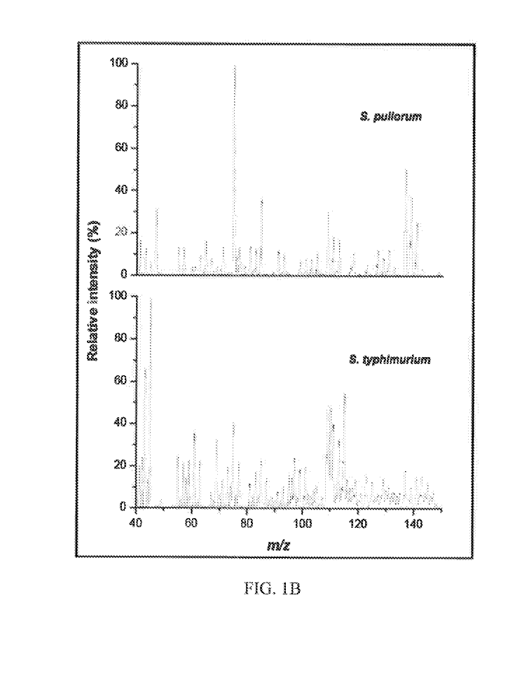

Traditional gas analysis involves some form of serial analysis; gas chromatography and mass spectrometry are well known, although there are many other analytical methods that generate a plot of a swept parameter (e.g., column residence time) and the measured intensity at each value of that parameter. Some of these methods can also be used to fractionate the sample, and that fractionated sample can then be subjected to secondary analyses. When each of these is treated as a value in a characterization vector, an n-dimensional characterization of the sample can be obtained. An example is the use of gas chromatography (“GC”) followed by a differential ion mobility analysis (“GC”) (collectively, “GC-DMS”). A schematic of such an instrument is illustrated in FIG. 2, and an example of an odorgram produced by the gas analyzer (also referred to herein as an odorgram analyzer) is shown in FIG. 3.

FIG. 2 illustrates a simplified block diagram of an electronic odor sensor 301 (also referred to herein as an “e-nose”). In the system 301-302, a gas chromatograph (“GC”) 304 may be coupled with a differential ion mobility spectrometer (“DMS”) 305, the combination also referred to as “GC-DMS.” Input gas 300 comes into the e-nose 301 through a port. In a configuration of the e-nose 301, the input gas 300 is passed through a trap 303 that concentrates the analytes (e.g., volatile organic compounds (“VOC”)) in the gas. Then the concentrated gas is passed through a GC column 304, The GC column 304 is then eluted into the differential mobility spectrometer (DMS) 305. The DMS 305 is part of a family of ion mobility spectrometers that is related to High-Field Asymmetric Waveform Ion Mobility Spectrometry (“FAIMS”) (see, e.g., Roger Guevremont, “High-Field Asymmetric Waveform Ion Mobility Spectrometry,” Canadian J. of Anal. Sciences and Spectroscopy, Vol. 49 (3), pp. 105-113, 2004, which is hereby incorporated by reference herein). Examples of tools that may be used to monitor analytes are gas chromatographs, gas chromatographs coupled to mass spectrometers, and was chromatographs coupled to ion mobility spectrometers. Ion mobility spectrometers may include time-of-flight spectrometers and FAIMS (Field Asymmetric Waveform Ion Mobility Spectrometry). In some cases, the mass spectrometer and/or the ion mobility spectrometer may be used independent of a gas chromatograph. In some cases, the mass spectrometer may be coupled with an ion mobility spectrometer. In some eases, a gas chromatograph may be coupled to both an ion mobility spectrometer and a mass spectrometer, either in series or in parallel. Embodiments of the present invention are not limited to using the foregoing as the odorgram analyzer, however.

Examination of the example odorgram in FIG. 3 shows that use of only one of the two analysis tools would not have separated several peaks into their overlapped constituent components. DMS alone would have not revealed the many analyst components in the positive 0-10 volt range that the GC was able to resolve. Similarly, use of the GC alone would not have revealed the many overlapping constituent analytes seen in the 100-500 minutes residence time range. Use of both GC and DMS together provides a much higher level of discrimination of the constituent analytes present in the sample.

Looking at just an odorgram, it is difficult to determine a priori which constituents represent a desired signal and which represent contaminants from the environmental background. Kwak and Preti, previously referenced, have illustrated the perniciousness of those contaminants in their critique paper. It is not just the ambient odors at the time the sample is collected, but any contaminants emanating from the subject and not related to the condition that are being tested for. For example, testing the breath of a patient for chemical signals of the onset of pneumonia can be confounded by the analytes absorbed by the patient from vehicle exhaust in route to the testing center. The body odor of human subjects is also a major source of volatile analytes. Currently, analytes can only be rejected as background (i.e., “noise”) if their chemical compounds can be identified as biochemically exogenous to the condition under test.

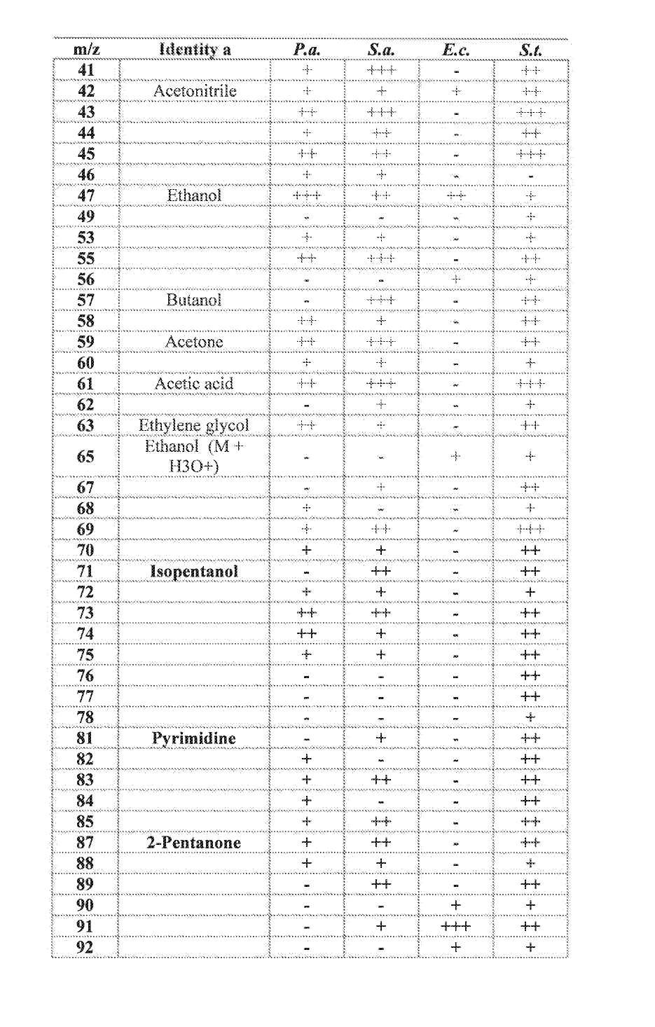

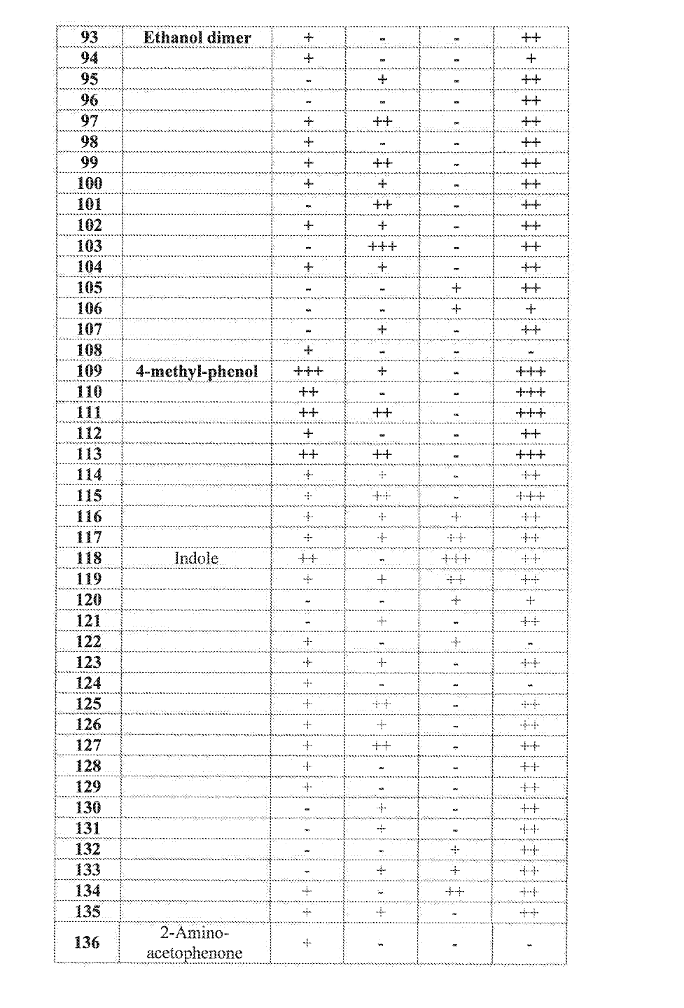

Further complicating the situation is that many of the constituents that can be detected with analytical instruments have not been identified or are not identifiable. Many such unknown constituents can be seen in the table in FIG. 1A. If they cannot or are not identified, then they cannot be excluded a priori from use as indicators of the condition under test. Unfortunately, the vast majority of published headspace analysis papers have focused on the identified/identifiable elements, leaving little scientific knowledge about the nature of these mystery constituents.

In the event that the desired odorgram of the targeted condition has been previously determined, by laboratory work or careful sampling in simulated field environments, a solution is to employ a correlation of field-acquired samples' odorgrams with the known desired odorgram and report a goodness-of-fit metric to the operator. A method would be the use of cross-correlation between the known odorgram and the field-acquired odorgram(s) to compute a correlation coefficient. Another approach is to use peak-matching or k-nearest-neighbor methods to quantitatively compare the two odorgrams. Prior knowledge of which regions of the odorgram are the most indicative of the target condition and which regions are the most prone to external contaminating constituents may be used to weight the comparisons.

However, determining the desired odorgram of the targeted condition can be quite difficult, because recreations of the desired aromas are likely to not fully represent those found under field circumstances. For example, the use of laboratory-incubated cultures of bacteria as a source of aromas indicative of infections will not be representative of infected wound aromas due to the differences in the bacterial substrates, agar instead of tissue. Further, it is also known that bacteria produce different aromas in different stages of growth, and therefore the odorgram of an early-stage infection may be, but is not assured to be, different from a late-stage infection.

A more complete solution, then, is to find a source of related, but not necessarily identical, constituent “noise” gas and cancel the presence of that noise from the target sample in order to arrive at a pure signal, regardless of its source or circumstance.

Adaptive noise cancellation was introduced by Bernard Widrow in the 1970's at the Naval Research Station in San Diego (see, Widrow et al., “Adaptive noise cancelling: principles and applications,” Proc IEEE, 63 (12):1692-1716, 1975, which is hereby incorporated by reference herein). The signal processing principles he used can be applied to solve the current problem by recognizing that the noise in question can be transformed to a digital domain once the reference source and the sample source gasses are converted to signals by the analyzer. Performed adaptively, this approach removes all components of noise from the final odorgram that are present in the odorgram of the admixture of signal and noise gas constituents.

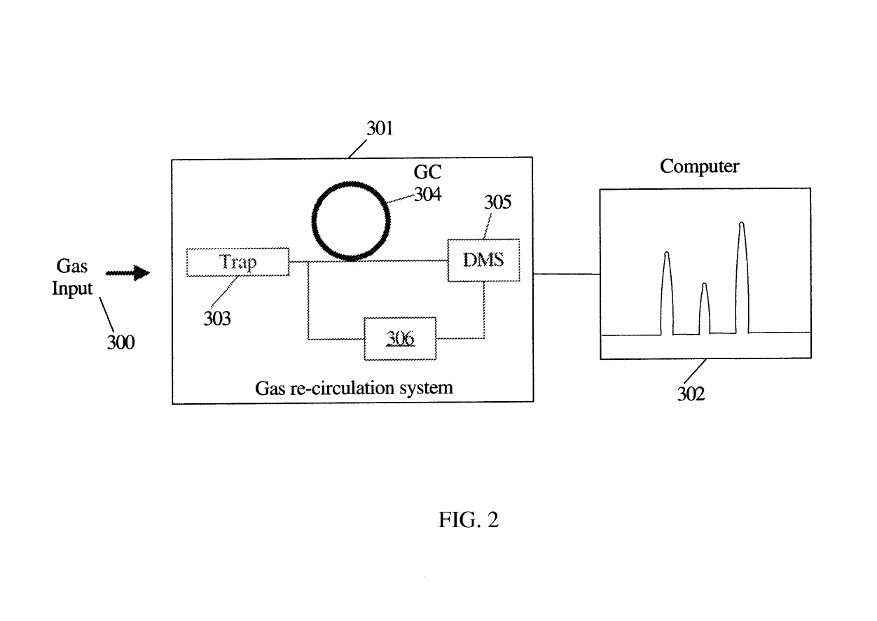

A schematic of a basic processing flow is illustrated in FIG. 4. G is the test sample of gas produced by the chemical process targeted for detection but also containing unknown constituents of background contamination. G′ is a reference sample that contains the environmental markers that are admixed into the test sample in unknown proportions. The odorgram analyzers may be GC-DMS devices, or any equivalent thereof. N′ is the odorgram produced by analysis of G′. S+N is the odorgram produced by analysis of G. F is an unknown transformation function that mimics the alterations that occur between G′ and G. N̂ is the modeled approximation of N′ obtained by transforming S+N with F. Subtracting N′ from N̂ yields an error estimate, Y, which is then fed back to incrementally and iteratively alter the model until a minimum error output is obtained. It is Y that represents the background-suppressed odorgram. Several such iteration algorithms exist in linear and nonlinear architectures, such as Widrow's LMS method previously noted. Because of the nonlinear nature of the sources of the constituent chemicals and the analyzers, the algorithm may be nonlinear, such as a radial basis function neural network. The source of the reference sample is only constrained by the requirement that it contain none of the “signal.” If there are constituents in the “signal” also present in the background, then they will be removed by the background suppression. Note that repeated samples of G′ allow the system to adapt its approximation of the contamination process over time.

Successful application of noise cancellation methods requires a reference source containing as little of the desired signal as possible. To obtain such a reference gas in the field will depend on the specific application to which the odor analyzer is put. In any case, it will require some specific apparatus to be built that will maximally exclude gas from the target source.

Recognizing Kwak and Preti's (see previous reference) objections to typical breath analysis as a valid concern for historical exposure to environmental trace contaminants, embodiments of the present invention utilize one or more of at least two basic solutions for the source of the reference gas. These solutions utilize an attribute of the adaptive noise canceller not requiring an exact copy of the contaminants present in the admixture sample but merely to be representative of those components.

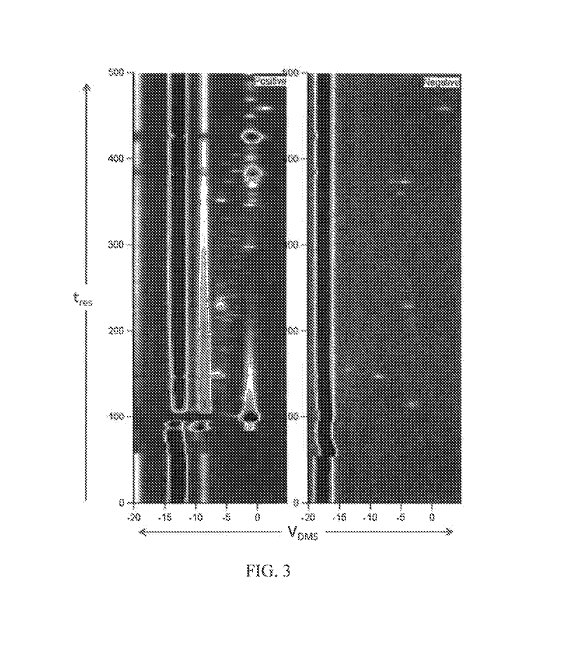

The embodiment illustrated in FIG. 4 utilizes a first odorgram analyzer for receiving the reference sample of gas G′ and outputting the N′ odorgram, and a second odorgram analyzer for receiving the test sample of gas G and outputting the S+N odorgram. Referring to FIG. 5, there is illustrated an alternative embodiment that multiplexes the analysis of gas samples G and G′ by using a single analyzer instrument (e.g., a GC-DIMS). Because these instruments are costly, it is impractical but not impossible to operate one for each channel of analysis. One advantage of using a single analyzer section is to avoid any differences in analysis sensitivity as is often seen between multiple instruments. A secondary sampling port inlet receives the reference sample of gas G that is then transferred via a valve to a single odor analyzer to generate the reference odorgram N′. After any needed resetting of functions on the analyzer have been completed to prepare it for the subsequent aliquot of gas, the valve is then switched to the other inlet port for sampling the admixture gas G, whereby the odorgram analyzer then analyzes the gas G and outputs the S+N odorgram. Thereafter, the odorgrams N′ and S+N are further analyzed as described with respect to FIG. 4.

The source of reference gas is likely to be abundant (e.g., from the ambient room air) whereas the admixture gas (e.g., drawn from a patient) may be only occasionally available. This is convenient for training the noise rejection transfer function F iteratively by repeatedly sampling the reference gas. The repeated samples of reference gas may be analyzed and used to generate updated versions of the reference odorgram N′ while processing and updating the transfer function F using the singular version of the admixture odorgram S+N. Iteration is often required of adaptation algorithms to cause the transfer function to converge to a stable solution. If serial samples of the reference gas are not available, then the odorgrams N′ and S+N may be synthetically dithered to provide the signal variance required to obtain convergence of the transfer function model.

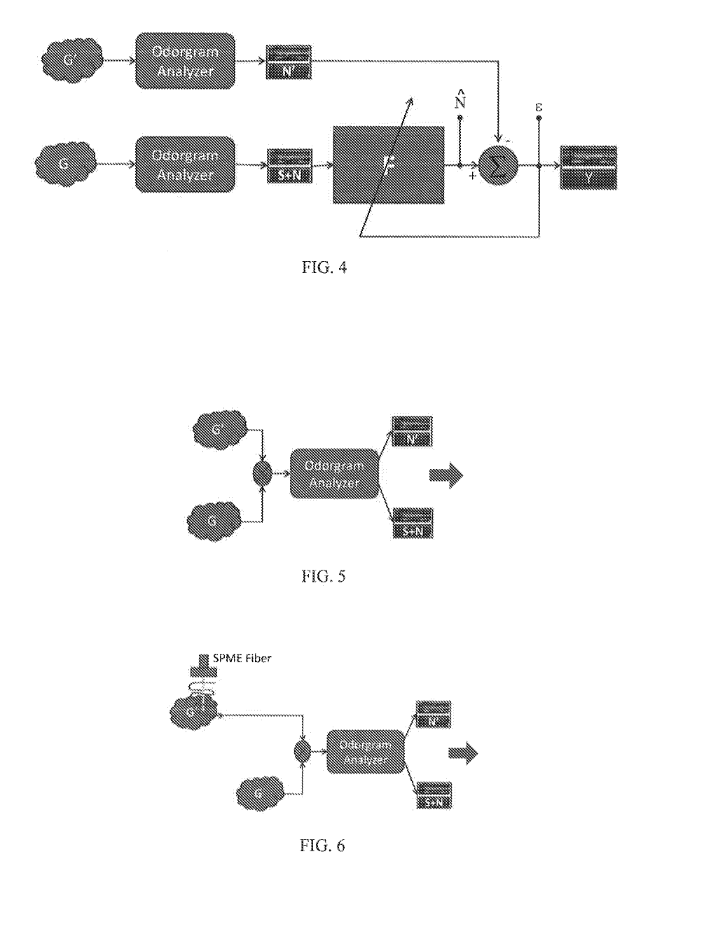

Retelling to FIG. 6, other embodiments of the present invention utilize an environmental sampler that travels with the subject being tested, and yet not be exposed to the signal-containing gasses produced by the subject. One such sampling device for trapping such gasses is known as a solid-phase microextraction (“SPME”) fiber. The, for sourcing the reference gas, the subject (e.g., a patient) utilizes the environmental sampler, or trap, (e.g., a SPME fiber) to carry with them during their daily activities in a protective but porous shell. The SPME fiber will accumulate the environmental constituents to which the subject is exposed and may then be used as a source of reference gas constituents to generate N′ that will contain residual constituents that are not present at the time of the drawing of the admixture sample. Thereafter, the odorgrams N′ and S+N are further analyzed as described with respect to FIG. 4.

It is possible to obtain the gas samples in real time or from trapping technologies. Use of a trap is especially advantageous for the reference sample in healthcare applications, as shown in this figure, because it mimics the accumulation of constituent chemicals presented to the individual patient over time and absorbed into their body. These absorbed compounds then are released in odor gas samples along with the markers of pathology that are sought in the test. Without use of a trap travelling with the patient during their daily living, these compounds, which are indeed chemical noise, can be applied at the noise reference input. Without sampling of these compounds, it would appear as if they were generated by the patient, i.e., markers of the pathology being tested for.

What is claimed is:

1. A system for analyzing an aroma emanating from a source, comprising:
a test sample of gas containing an unknown chemical composition produced by the source;
a reference sample of gas containing an unknown background chemical that is not produced by the source;
one or more gas analyzers suitable for receiving the test sample of gas and the reference sample of gas and outputting it least a two-dimensional odorgram pertaining to the received test sample of gas and the reference sample of gas; and
an iterative adaptive function implemented as a software program operating on a computer, the iterative adaptive function suitable for suppressing the unknown background chemical from the odorgram so that an identity of the unknown chemical composition can be determined.

2. The system as recited in claim 1, wherein the one or more gas analyzers comprise a GC-DMS analyzer.

3. The system as recited in claim 1, wherein the reference sample of gas does not contain any chemicals produced by the source.

4. The system as recited in claim 3, wherein the test sample of gas contains a mixture of the unknown chemical composition and the unknown background chemical.

5. The system as recited in claim 4, wherein the one or more gas analyzers comprise, a first GC-DMS analyzer suitable for receiving the test sample of gas, and a second GC-DMS analyzer suitable for receiving, the reference sample of gas, wherein the first GC-DMS analyzer outputs a first odorgram as a function of an analysis of the test sample of gas, and wherein the second GC-DMS analyzer outputs a second odorgram as a function of an analysis of the reference sample of gas.

6. The system as recited in claim 5, wherein the software program is further suitable for subtracting the second odorgram from a modeled approximation of the second odorgram to produce a background-suppressed odorgram that indicates the identity of the unknown chemical composition.

7. The system as recited in claim 1, further comprising a trap for collecting the reference sample of gas.

8. The system recited in claim 7, wherein the trap is a SPME fiber.

9. The system as recited in claim 1, wherein the source is a human, and the test sample of gas comprises the unknown chemical composition produced as an aroma by the human.

10. A method for analyzing an aroma emanating from a source, comprising:
receiving by a first gas analyzer a test sample of gas containing an unknown chemical composition produced by the source;
receiving by a second gas analyzer a reference sample of gas containing an unknown background chemical that is not produced by the source,
wherein the test sample of gas contains a mixture of the unknown chemical composition and the unknown background chemical;
outputting from the first gas analyzer a first odorgram as a function of an analysis of the test sample of gas;
outputting from the second gas analyzer a second odorgram as a function of an analysis of the reference sample of gas; and
suppressing the unknown background chemical from the first odorgram so that an identity of the unknown chemical composition can be determined.

11. The method as recited in claim 10, wherein the first and second gas analyzers are a single gas analyzer.

12. The method as recited in claim 10, wherein the reference sample of gas does not contain any chemicals produced by the source.

13. The method as recited in claim 10, wherein the first gas analyzer comprises a first GC-DMS analyzer suitable for receiving the test sample of gas, and wherein the second gas analyzer comprises a second GC-DMS analyzer suitable for receiving the reference sample of gas.

14. The method as recited in claim 10, wherein suppressing the unknown background chemical from the first odorgram further comprises subtracting the second odorgram from a modeled approximation of the second odorgram to produce a background suppressed odorgram that indicates the identity of the unknown chemical composition.

15. The method as recited in claim 10, wherein receiving by the second gas analyzer the reference sample of as further comprises collecting the reference sample of gas with SPME fiber.

16. The method as recited in claim 10, wherein the source is a human, and the test sample of gas comprises the unknown chemical composition produced as an aroma by the human.

17. The method as recited in claim 16, wherein the reference sample of gas is received by the the second gas analyzer from an environment surrounding the human at a time when the test sample of gas is collected by the the first gas analyzer.
